(19) United States
(12) Patent Application Publication
(10) Pub. No.: US 2021/0244341 A1
(43) Pub. Date: Aug. 12, 2021

(54) METHOD OF PROVIDING VENTRICULAR ARRHYTHMIA LOCALIZATION WITH A HEART MODEL DERIVED FROM MACHINE LEARNING

(71) Applicant: CATHETER PRECISION, INC., Mt. Olive, NJ (US)

(72) Inventor: Barry YOMTOV, Marblehead, MA (US)

(21) Appl. No.: 17/174,328

(22) Filed: Feb. 11, 2021

Related U.S. Application Data
(60) Provisional application No. 62/975,114, filed on Feb. 11, 2020.

(51) Int. Cl. A61B 5/363; A61B 5/343; G16H 50/50; G16H 10/60; G16H 30/40; G16H 50/30

(52) U.S. Cl. CPC A61B 5/363; A61B 5/343; G16H 50/30; G16H 10/60; G16H 30/40; G16H 50/50

(57) ABSTRACT

Various embodiments include methods and computing systems for arrhythmia localization and display. A computing system may select a 3D heart electrical conduction model, including a 3D surface model, from a database of representative 3D heart models based on patient demographic information. The computing system may generate a patient-specific 3D localization of an arrhythmia based on the selected 3D electrical conduction model and ECG data, and generate a patient-specific cardiac activation map based the 3D electrical conduction model and ECG data. The computing system may then merge the patient-specific 3D localization of the arrhythmia and the 3D surface model to generate a 3D arrhythmia activation surface model, and display the patient-specific 3D localization of the arrhythmia and the patient-specific cardiac activation map for use in a medical procedure. Patent demographic information may be used to create or adjust a 3D heart model for inclusion in the database.

### CROSS-REFERENCE TO RELATED APPLICATION

This application claims the benefit of priority to U.S. Provisional Patent Application No. 62/975,114, entitled “Method of Providing Ventricular Arrhythmia Localization with a Heart Model Derived from Machine Learning”, filed Feb. 11, 2020, the entire contents of which are hereby incorporated by reference for all purposes.

### BACKGROUND

Some heart defects in the conduction system result in asynchronous contraction (arrhythmia) of the heart and are sometimes referred to as conduction disorders. As a result, the heart does not pump enough blood, which may ultimately lead to heart failure. Conduction disorders can have a variety of causes, including age, heart (muscle) damage, medications and genetics.

Premature Ventricular Contractions (PVCs) are abnormal or aberrant heart beats that start somewhere in the heart ventricles rather than in the upper chambers of the heart as with normal sinus beats. PVCs typically result in a lower cardiac output as the ventricles contract before they have had a chance to completely fill with blood. PVCs may also trigger Ventricular Tachycardia (VT or V-Tach).

Ventricular tachycardia (VT or V-Tach) is another heart arrhythmia disorder caused by abnormal electrical signals in the heart ventricles. In VT, the abnormal electrical signals cause the heart to beat faster than normal, usually more than 100 beats per minute, with the beats starting in the heart ventricles. VT generally occurs in people with underlying heart abnormalities. VT can sometimes occur in structurally normal hearts, and in such patients the origin of abnormal electrical signals can be in multiple locations in the heart. One common location is in the right ventricular outflow tract (RVOT), which is the route the blood flows from the right ventricle to the lungs. In patients who have had a heart attack, scarring from the heart attack can create a milieu of intact heart muscle and a scar that predisposes patients to VT.

### SUMMARY

Various embodiments include methods and computing systems implementing the methods for arrhythmia localization and display for use in a medical procedure. Various embodiments may include selecting a three-dimensional (3D) heart electrical conduction model from a database of representative 3D heart models based on patient demographic information, the selected 3D heart electrical conduction model including a 3D surface model, generating a patient-specific 3D localization of an arrhythmia based on the selected 3D electrical conduction model and electrocardiographic (ECG) data, generating a patient-specific cardiac activation map based the 3D electrical conduction model and ECG data, merging the patient-specific 3D localization of the arrhythmia and the 3D surface model to generate a 3D arrhythmia activation surface model, and displaying the patient-specific 3D localization of the arrhythmia and the patient-specific cardiac activation map for use in a medical procedure. In various embodiments, the patient demographic information may include one or more of the patient's gender, age, weight, height, body mass index, waist circumference, chest circumference, or underlying etiology.

Some embodiments may further include generating a patient-specific 3D heart model from the selected 3D heart model by obtaining a 3D image of ECG electrodes on the patient's torso, and merging the 3D image of the patient's torso with the selected 3D heart model.

In some embodiments, merging the 3D image of the patient's torso with the 3D patient specific heart model may include aligning locations of ECG electrodes used in generating patient-specific electrical conduction map of a patient's heart with the ECG electrodes within the 3D image. In some embodiments, ECG data obtained with 12 ECG electrodes may be combined with the patient specific 3D heart model using an inverse solution calculation to generate a localization point of the arrhythmia activation in a heartbeat.

In some embodiments, the arrhythmia may be a ventricular arrhythmia In some embodiments, the ventricular arrhythmia may be a PVC. In some embodiments, the ventricular arrhythmia may be a ventricular tachycardia.

Some embodiments may further include using the patent's demographic information and the patient-specific 3D heart model to create a new 3D heart model for inclusion in the database of representative 3D heart models. Some embodiments may further include using the patent's demographic information and the patient-specific 3D heart model to adjusting a 3D heart model in the database of representative 3D heart models.

Further embodiments include a computing system having a memory storing a database of representative 3D heart models, and a processor coupled to the memory and configured with processor-executable instructions to perform operations of any of the embodiments summarized above.

### BRIEF DESCRIPTION OF THE DRAWINGS

The accompanying drawings, which are incorporated herein and constitute part of this specification, illustrate example embodiments of the invention, and together with the general description given above and the detailed description given below, serve to explain the features of the invention.

FIG. 1 is a system block diagram of a computing system configured to perform operations of various embodiments.

FIG. 2 is an example of a 3D heart model of the patient specific heart model that may be stored within the heart model data base according to various embodiments.

FIG. 3 illustrates example results of a localization and activation map of a single arrhythmia event.

FIG. 4 illustrates an example operational workflow for an embodiment method four generating a localization of an arrhythmia

FIG. 5 is a system block diagram of a computing system configured to perform operations of a second embodiment.

FIG. 6 illustrates an example operational workflow for an embodiment method for generating a localization of an arrhythmia according to the second embodiment.

FIGS. 7A-7C are process flow diagrams illustrating methods for generating arrhythmia activation surface models suitable for use in a medical procedure according to some embodiments.

FIG. 8 is a component block diagram illustrating an example mobile computing device suitable for use with the various embodiments.

FIG. 9 is a component block diagram illustrating an example server suitable for use with the various embodiments.

### DETAILED DESCRIPTION

The various embodiments will be described in detail with reference to the accompanying drawings. References made to particular examples and implementations are for illustrative purposes, and are not intended to limit the scope of the invention or the claims.

Catheter ablation is a treatment of choice in patients with VT and/or symptomatic PVCs. The targets for ablation are locations in the heart where PVCs are occurring or locations where the onset of the VT is occurring. In order to determine a proper ablation location, a treating physician may first stimulate a proposed location using an electrical lead, in order to determine whether ablation at the proposed location will provide a desired electrical activation pattern stimulation of the heart. At times, there is a need to determine where the area of earliest activation (or localization) of the PVC within the heart's myocardium.

To aid a clinician in performing an electrophysiology procedure, such as an ablation treatment, a computer system may use imaging data from a magnetic resonance imaging (MRI) or computed tomography (CT) scan to generate a patient-specific 3D heart model. In some less developed countries, imaging systems may not be readily available at hospitals. To address this reality and for cost reduction, various embodiments include methods that make use of a database of representative 3D heart and torso models to generate a patient-specific PVC activation model without the need for generating a 3D heart model based on MRI or CT image data. In various embodiments, a heart and torso model consistent with the patient's body may be selected from a series of representative 3D models maintained within a 3D heart model database. In some embodiments, the 3D heart model may be selected automatically by a computer system based on a patient demographics, such as gender, height, weight and/or body measurements, as well as health conditions or underlying etiology.

As used herein, an electrocardiogram (ECG) is any method that (preferably non-invasively) correlates actual electrical activity of the heart muscle to measured or derived (electrical activity) of the heart. In case of a classical electrocardiogram, the differences in potential between electrodes on the body surface are correlated to the electrical activity of the heart. In order to obtain a functional image, an estimation of the movement of the electrical activity may be provided. As used herein, the term “electrocardiographic data” refers to graphical information related to or displaying ECG data.

Various embodiments include methods of identifying and displaying the location in the heart of the earliest activation of an arrythmia, which is referred to herein as “localization” of an initiation site of the arrhythmia. The methods of arrhythmia localization and heart modeling may be performed by a computing system. Various embodiments may include generating a patient specific electrical conduction map of a heart during an arrhythmia based on a 3D heart model and electrocardiogram (ECG) data, such as ECG data recorded by a 12-lead ECG unit while the patient experiences an arrhythmia event. Various embodiments may enable the development of patient-specific heart models based on the electrical conduction map without the need for MRI or CT image data by selecting a 3D model of the heart from a database of representative 3D heart models based on the patient's demographic information. Examples of patient demographic information that may be used for selecting a suitable representative 3D heart model from the database may include one or more of the patient's gender, age, weight, height, body mass index, waist circumference, chest circumference, and/or underlying etiology. A patient-specific arrhythmia localization model may then be generated by the computing system by merging a 3D localization of the arrhythmia and a 3D internal surface model that is part of the selected representative 3D heart model to form an arrhythmia activation surface model. The computing system may then display the arrhythmia activation surface model or produce an output of the arrhythmia activation surface model for display to a clinician for use in a medical procedure. For example, the arrhythmia activation surface model may be displayed to guide a clinician performing an electrophysiology procedure (e.g., an ablation procedure) to treat the arrythmia.

Some embodiments may include generating a patient specific 3D heart model from a 3D model database. In some embodiments, a 3D image may be taken of the ECG electrodes of the patient's torso, and the 3D image of the patient's torso may be merged with the selected 3D patient specific heart and torso model, which also includes modeled locations of ECG electrodes. In this process, the locations of the ECG electrodes within the selected 3D heart and torso model may be aligned with the ECG electrodes visible within the 3D image of the patient's torso. In some embodiments, 12 ECG data may be combined with the patient specific 3D heart model using an inverse solution calculation to generate a localization point of the arrhythmia initiation point (e.g., PVC activation) within heartbeats.

The embodiment methods may be useful for treating arrhythmia that are a ventricular arrhythmia, which may be a PVC or a ventricular tachycardia. The embodiment methods may also be useful for treating other forms of arrhythmia, such as an atrial arrhythmia or a dysrhythmia between the two ventricles.

In some embodiments, machine learning may be used to create a new 3D heart and torso model based on recorded patient demographic information. In some embodiments, machine learning based on continued collection of patient demographic data may be used to adjust representative 3D heart models in the database of representative 3D heart models.

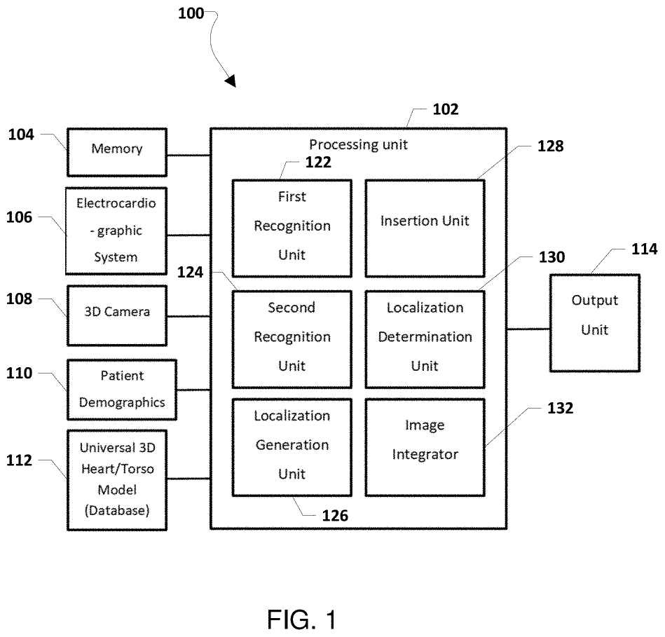

FIG. 1 is block diagram providing a schematic representation of an example computing system 100 for providing a representation of ECG localization of arrythmias within heart tissue. The computing system 100 may include a processing unit 102, a memory 104, an electrocardiographic system 106, a 3D camera 108, storage or an input of patient demographic information 110, a universal 3D heart and torso model 112, and an output unit 114, such as a display or a network connection for outputting display dated to a remote display unit (not shown). Input data is provided from an electrocardiographic (ECG) system 106, and a picture from a 3D camera 108. The processing unit 102 may include a first recognition unit 122, a second recognition unit 124, a localization generation unit 126, and insertion unit 128, a localization determination unit 130, and an image integrator 132. These units may be separate processing systems, software modules of processor-executable instructions configured to be executed in a single processor within the processing unit, dedicated hardware, or combinations thereof.

The 3D localization of the ECG within the heart tissue may be obtained by the processing unit 102 combining electrocardiographic data from the electrocardiographic system 106 with a universal 3D heart and torso model from the database that is selected from the database of 3D heart models 112 based on the patient's demographic information 110. This data may be stored in the memory 104 or input via various types of input devices, such as a keyboard or a network connection (not shown). The processing unit 102 may be connected to the electrocardiographic system 106 and the 3D camera 108 for retrieving the data and storing corresponding data in the memory 104.

An electrocardiographic imaging (ECGI) method able to determine the localization of the initiation site of arrhythmias within the heart tissue from a 12 lead ECG may be applied by the processing unit 102 for determining the localization of arrhythmias. The ECG signals may be combined by the processing unit 102 with a patient-specific (adapted) 3D anatomical model of the heart and torso in order to compute the positions of the cardiac isochrones. The patient-specific 3D anatomical model may be automatically selected by the processing unit 102 from a universal heart/torso model database 112 based on a patient demographics 110, for example gender, height, body mass index (BMI), and/or weight.

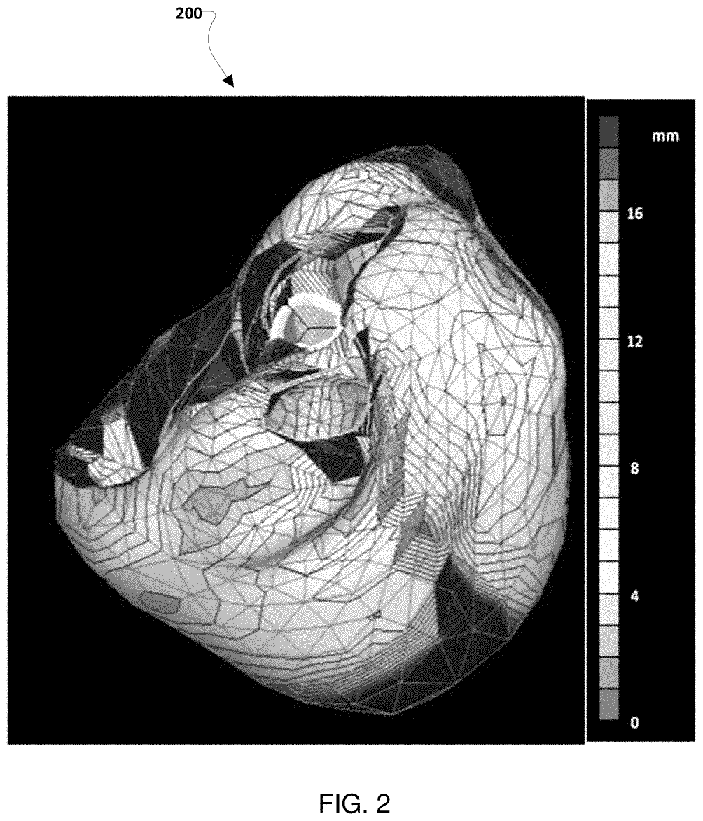

FIG. 2 illustrates an example of a representative 3D heart model 200 that may be stored within the database of representative 3D heart models. In FIG. 2, the scale represents the thickness of the heart wall (i.e., the myocardium) in mm The lighter shades, which could be displayed in light color, represent thinner myocardium and the darker shades, which could be displayed in darker (e.g., green to blue) colors, represent the thicker myocardium.

The characteristics (e.g., size, shape, myocardium wall thickness, etc.) of the 3D heart model may be based on patient features and demographic information, such as gender, weight, height and BMI. Each feature may have a weighted factor that the computing system may utilize in determining selecting the appropriate representative 3D heart model to use to generate a patient-specific arrhythmia activation 3D heart model. Each weighted factor may have a relationship to the characteristics of each 3D heart model in the database, such as the heart size (e.g. left and right ventricles), shape, overall dimensions, and/or myocardium wall thickness. Additional demographics may also include the patient's underlying heart conditions (e.g. enlarged left ventricle). Such a condition would impact the size of the left ventricle from the 3D heart model database.

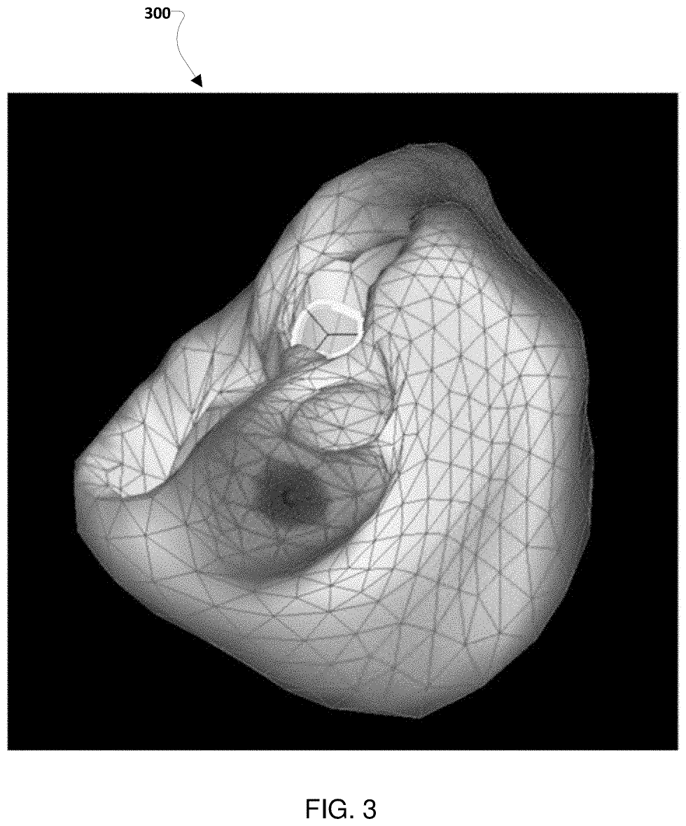

FIG. 3 illustrates example results of a localization and activation map of a single arrhythmia event, such as a PVC event, that may be produced by a computing system 100 implementing various embodiment methods. In the illustrated example, the localization point is on the epicardial surface of the myocardium displayed as a dot within a surrounding circular shaded region. The shading gradient illustrates the activation of the PVC heartbeat during the duration of the QRS complex.

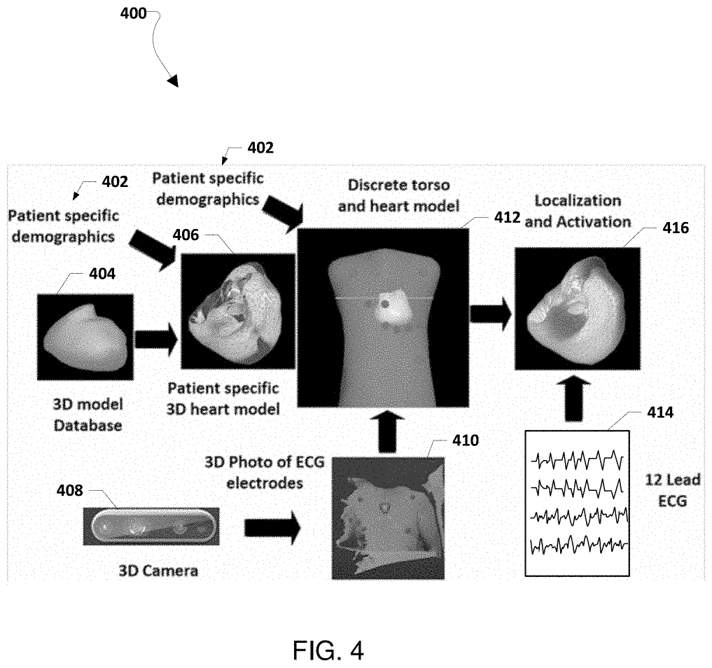

FIG. 4 illustrates an example operational workflow 400 for a method of generating the localization of an arrhythmia, such as a PVC, and for superimposing the localization on the patient specific 3D heart model including the myocardium wall thickness.

A heart model from a predetermined database 402 of representative 3D heart models, for example ten (10) such models within a universal 3D heart model database 404, may be selected as a patient specific 3D heart model based on patient demographics 402 specific to the patient. These patient specific demographics 402 may include for example, gender, race, BMI, height, waist chest circumference, underlying etiology, etc. The heart models within the database 404 may be inclusive of calculations for the inverse solution for the localization of the arrhythmia, such as a PVC, from an ECG. A patient specific torso model 412 may also the selected from the heart and torso model data base 404, and inclusive of such models.

A 3D camera 408 may be used to take a 3D image 410 of the patient's torso inclusive of ECG electrodes used for a 12-lead electrocardiograph recording 414. The 3D image 410 may be merged with the patient specific 3D heart and torso model 412, with the ECG electrodes aligned between the 3D image 410 and electrode positions in the torso model 412. ECG markers may be placed in certain locations within and arrhythmia heartbeat, such as within the QRS waveform of a PVC. A mathematical model may be used for generating the inverse solution from the 12 lead ECG recording 414 within the timing of the ECG markers to calculate the localization and activation of arrhythmias (e.g., a PVC) and generating a cardiac activation map 416.

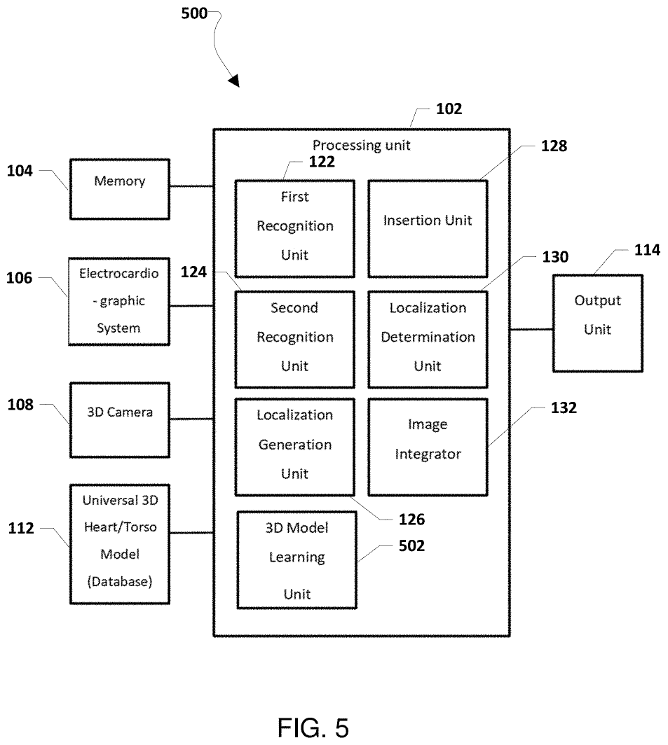

In a second embodiment, the computing system 500 may also include a 3D Model Learning Unit 502 as illustrated in FIG. 5. The computing system 500 may include the same components and units as in the computing system 100 as described with reference to FIG. 1. Adding a 3D model learning unit 502 to the processing unit 102 enables the computing system 500 to create new 3D heart models to be added to the database of representative 3D heart models 112, as well as to update or adjust the selected 3D heart models in the database based on patient demographic data and/or patient-specific 3D heart models generated according to various embodiments. As patients continue to be tested, the representative 3D heart models in the database 112 may be refined to accommodate the patient demographics that are stored in memory 104. The 3D model learning unit 502 may sort the patient demographics as part of such operations. In this manner, the database of representative 3D heart models 112 can be expanded and improved over time based on experience gained from numerous patients.

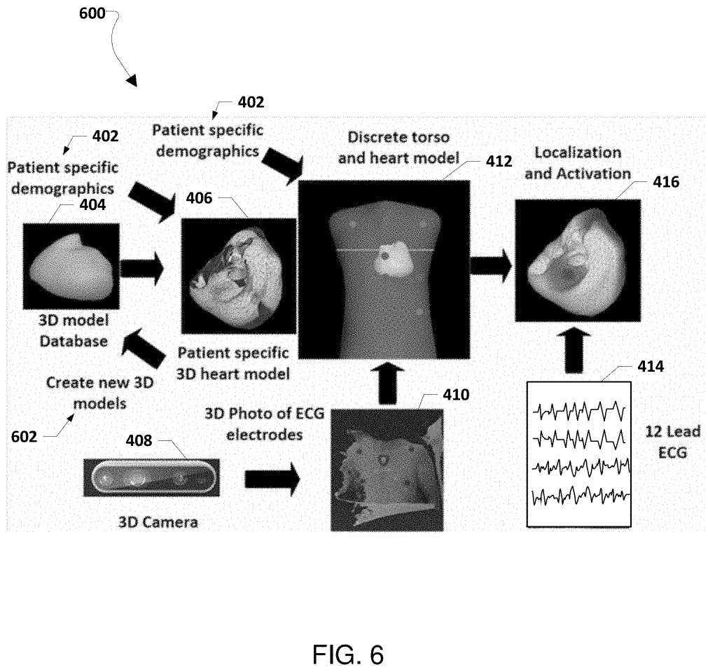

FIG. 6 illustrates an example operational workflow 600 of a second embodiment method of generating localization of a PVC based on the 3D database heart model 404 including a feedback process for learning patient demographic data 402 and creating new 3D heart models based on this patient data. The operational workflow 600 may include the operations 402-416 of the operational workflow 400 described with reference to FIG. 4. In addition, adjustments or adaptation of the 3D heart model or the creation of additional heart models (collectively 602) may be performed based on the collection of statistics of patient demographic data gathered during ongoing testing of patients. For example, if through continued testing, one of the patient demographics data items, such as patient weight, had a skewed distribution towards heavier patients, a 3D heart model characteristic (for example wall thickness) may be adjusted to generate a new heart model for the 3D heart model database.

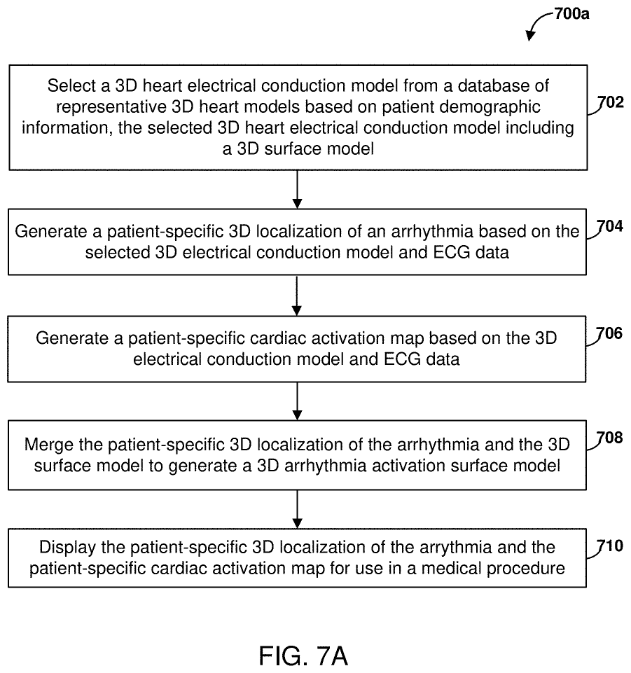

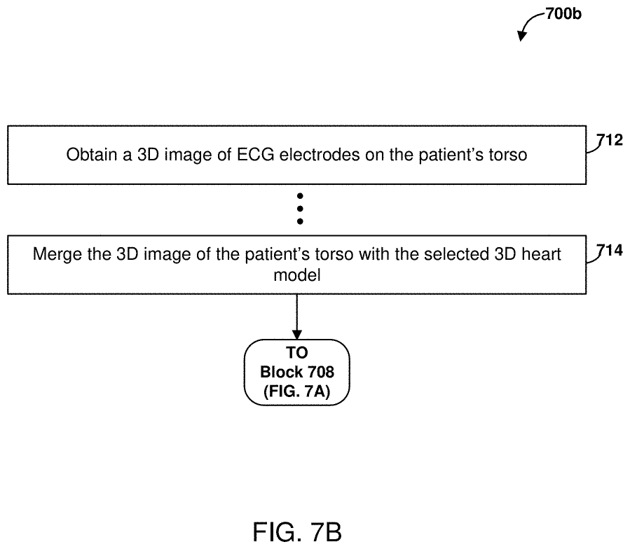

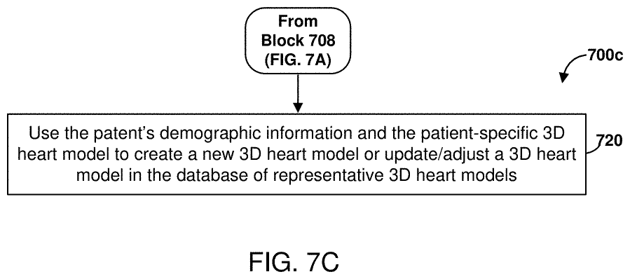

FIG. 7A is a process flow diagram illustrating an embodiment method 700a for determining a likelihood of success and/or complications from performing an ablation procedure to cure arrhythmia at a particular location on the heart. The operations of the method 700a may be performed by one or more processors within a processing unit (e.g., 102) of a computing system (e.g., 100, 500).

In block 702, the processing unit 102 may a 3D heart electrical conduction model from a database of representative 3D heart models based on patient demographic information. The selected 3D heart electrical conduction model may include a 3D surface model useful for showing the location of arrhythmia initiation sites. The patient demographic information used to select an appropriate 3D heart model may include one or more of the patient's gender, age, weight, height, body mass index, waist circumference, chest circumference, or underlying etiology, as well as other demographic and patient-specific information.

In block 704, the processing unit 102 may generate a patient-specific 3D localization of an arrhythmia based on the selected 3D electrical conduction model and ECG data. The generated patient-specific electrical conduction model of the patient's heart identifies the localization of an initiation site of the arrhythmia, i.e., the location in the heart of the earliest activation of an arrythmia

In block 706, the processing unit 102 may generate a patient-specific cardiac activation map based the 3D electrical conduction model and ECG data. In some embodiments. In some embodiments, this operation may be accomplished with ECG data obtained with 12 ECG electrodes using an inverse solution calculation to generate a localization point of the arrhythmia activation in a heartbeat.

In block 708, the processing unit may merge the patient-specific 3D localization of the arrhythmia and the 3D surface model to generate a 3D arrhythmia activation surface model.

In block 710, the processing unit may display or otherwise output the patient-specific 3D localization of the arrhythmia and the patient-specific cardiac activation map for use in a medical procedure. In some embodiments, the processing unit may provide an output suitable for rendering a visual display on a remote display unit for use in a medical procedure. For example, the arrhythmia activation surface model may be displayed to a clinician performing an ablation procedure to treat the patient's arrhythmia Examples of medical procedures in which the display may be used include an electrophysiology procedure, such as an ablation therapy.

The operations in the method 700a may be useful for generating arrhythmia activation surface models or a variety of different forms of arrhythmia, including ventricular arrhythmias, pre-ventricular contractions (PVC), ventricular tachycardia, an atrial arrhythmia or a dysrhythmia between the two ventricles.

FIG. 7B is a process flow diagram illustrating an example of operations of a method 700b that may be performed to generate a patient-specific 3D heart model useful in generating the arrhythmia activation surface model in block 708 of the method 700a (FIG. 7A). The operations of the method 700b may be performed by one or more processors within a processing unit (e.g., 102) of a computing system (e.g., 100, 500).

In block 712, the processing unit 102 may obtain a 3D image of the ECG electrodes on the patient's torso. This 3D image provides the processing unit with information regarding the orientation and dimensions of the patient's torso as well as the specific locations of each ECG electrode that collect the ECG data used in operations of block 702. The processing unit 102 may obtain the 3D image of the patient's torso during recording of ECG data, but the 3D image may also be taken after the ECG electrodes are attached but before the ECG data is recorded, or after the ECG data has been recorded.

In block 714, the processing unit 102 may merge the 3D image of the patient's torso with the representative 3D heart model selected from the 3D heart model database. In some embodiments, this operation of merging the 3D image of the patient's torso with the selected 3D heart model may include aligning the locations of ECG electrodes that are included in the selected 3D heart model with the ECG electrodes within the 3D image. In this manner, the processing unit 102 may generate a patient-specific 3D heart model using the selected representative 3D heart model as a starting point.

FIG. 7C is a process flow diagram illustrating an example of operations of a method 700c that may be performed to create or update/adjust representative 3D heart models based on patient demographic data. The operations of the method 700c may be performed by one or more processors within a processing unit (e.g., 102) of a computing system (e.g., 100, 500).

After an arrhythmia activation surface model is generated in block 708 of the method 700a, the processing unit may use the patient's demographic information in the patient-specific 3D heart model to create a new heart model or update/adjust a 3D heart model in the database of representative 3D heart models in block 720.

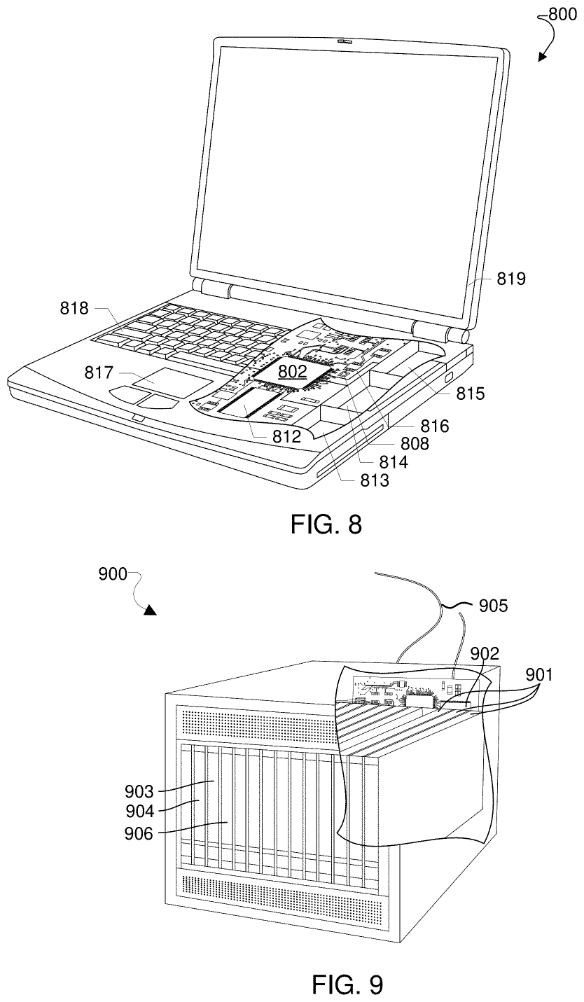

The various embodiments (including, but not limited to, embodiments described above with reference to FIGS. 1-7) may be implemented in a wide variety of computing systems include a laptop computer 800, an example of which is illustrated in FIG. 8. Many laptop computers include a touchpad touch surface 817 that serves as the computer's pointing device. A laptop computer 800 will typically include a processor 802 coupled to volatile memory 812 and a large capacity nonvolatile memory, such as a disk drive 813 or FLASH memory. Additionally, the computer 800 may have one or more antenna 808 for sending and receiving electromagnetic radiation that may be connected to a wireless data link (e.g., Bluetooth or Wi-Fi) and/or cellular telephone transceiver 816 coupled to the processor 802. The computer 800 may also include a floppy disc drive 814 and a compact disc (CD) drive 815 coupled to the processor 802. In a notebook configuration, the computer housing includes the touchpad 817, the keyboard 818, and the display 819 all coupled to the processor 802. Other configurations of the computing device may include a computer mouse or trackball coupled to the processor (e.g., via a USB input) as are well known, which may also be used in conjunction with the various embodiments.

The various embodiments (including, but not limited to, embodiments described above with reference to FIGS. 1-7) may also be implemented in fixed computing systems, such as any of a variety of commercially available servers. An example server 900 is illustrated in FIG. 9. Such a server 900 typically includes one or more multicore processor assemblies 901 coupled to volatile memory 902 and a large capacity nonvolatile memory, such as a disk drive 904. As illustrated in FIG. 9, multicore processor assemblies 901 may be added to the server 900 by inserting them into the racks of the assembly. The server 900 may also include a floppy disc drive, compact disc (CD) or digital versatile disc (DVD) disc drive 906 coupled to the processor 901. The server 900 may also include network access ports 903 coupled to the multicore processor assemblies 901 for establishing network interface connections with a network 905, such as a local area network coupled to other broadcast system computers and servers, the Internet, the public switched telephone network, and/or a cellular data network.

The foregoing method descriptions and the process flow diagrams are provided merely as illustrative examples and are not intended to require or imply that the steps of the various embodiments must be performed in the order presented. As will be appreciated by one of skill in the art the order of steps in the foregoing embodiment methods may be performed in any order.

The foregoing method descriptions and the process flow diagrams are provided merely as illustrative examples and are not intended to require or imply that the steps of the various embodiments must be performed in the order presented. As will be appreciated by one of skill in the art the order of steps in the foregoing embodiments may be performed in any order. Words such as “thereafter,” “then,” “next,” etc. are not intended to limit the order of the steps; these words are simply used to guide the reader through the description of the methods. Further, any reference to claim elements in the singular, for example, using the articles “a,” “an” or “the” is not to be construed as limiting the element to the singular.

The various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present invention.

The hardware used to implement the various illustrative logics, logical blocks, modules, and circuits described in connection with the aspects disclosed herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor may be a microprocessor, but, in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration. Alternatively, some steps or methods may be performed by circuitry that is specific to a given function.

In one or more exemplary aspects, the functions described may be implemented in hardware, software, firmware, or any combination thereof If implemented in software, the functions may be stored as one or more instructions or code on a non-transitory computer-readable medium or non-transitory processor-readable medium. The steps of a method or algorithm disclosed herein may be embodied in a processor-executable software module and/or processor-executable instructions, which may reside on a non-transitory computer-readable or non-transitory processor-readable storage medium. Non-transitory server-readable, computer-readable or processor-readable storage media may be any storage media that may be accessed by a computer or a processor. By way of example but not limitation, such non-transitory server-readable, computer-readable or processor-readable media may include RAM, ROM, EEPROM, FLASH memory, CD-ROM or other optical disk storage, magnetic disk storage or other magnetic storage devices, or any other medium that may be used to store desired program code in the form of instructions or data structures and that may be accessed by a computer. Disk and disc, as used herein, includes compact disc (CD), laser disc, optical disc, digital versatile disc (DVD), floppy disk, and Blu-ray disc where disks usually reproduce data magnetically, while discs reproduce data optically with lasers. Combinations of the above are also included within the scope of non-transitory server-readable, computer-readable and processor-readable media. Additionally, the operations of a method or algorithm may reside as one or any combination or set of codes and/or instructions on a non-transitory server-readable, processor-readable medium and/or computer-readable medium, which may be incorporated into a computer program product.

The preceding description of the disclosed embodiments is provided to enable any person skilled in the art to make or use the present invention. Various modifications to these embodiments will be readily apparent to those skilled in the art, and the generic principles defined herein may be applied to other embodiments without departing from the scope of the claims. Thus, the present invention is not intended to be limited to the embodiments shown herein but is to be accorded the widest scope consistent with the following claims and the principles and novel features disclosed herein.

What is claimed is:

1. A method of arrhythmia localization, comprising:
selecting a 3D heart electrical conduction model from a database of representative 3D heart models based on patient demographic information, the selected 3D heart electrical conduction model including a 3D surface model;
generating a patient-specific 3D localization of an arrhythmia based on the selected 3D electrical conduction model and electrocardiographic (ECG) data;
generating a patient-specific cardiac activation map based the 3D electrical conduction model and ECG data;
merging the patient-specific 3D localization of the arrhythmia and the 3D surface model to generate a 3D arrhythmia activation surface model; and
displaying the patient-specific 3D localization of the arrhythmia and the patient-specific cardiac activation map for use in a medical procedure.

2. The method of claim 1, wherein the patient demographic information includes one or more of the patient's gender, age, weight, height, body mass index, waist circumference, chest circumference, or underlying etiology.

3. The method of claim 1, further comprising generating a patient-specific 3D heart model from the selected 3D heart model by:
obtaining a 3D image of ECG electrodes on the patient's torso; and
merging the 3D image of the patient's torso with the selected 3D heart model.

4. The method of claim 3, wherein merging the patient-specific 3D localization of the arrhythmia and the 3D surface model to generate a 3D arrhythmia activation surface model comprises aligning locations of ECG electrodes used in generating patient-specific electrical conduction map of a patient's heart with the ECG electrodes within the 3D image.

5. The method of claim 4, wherein ECG data obtained with 12 ECG electrodes is combined with the patient specific 3D heart model using an inverse solution calculation to generate a localization point of the arrhythmia activation in a heartbeat.

6. The method of claim 4, wherein the arrhythmia is a ventricular arrhythmia

7. The method of claim 6, wherein the ventricular arrhythmia is a pre-ventricular contraction (PVC).

8. The method of claim 6, wherein the ventricular arrhythmia is a ventricular tachycardia.

9. The method of claim 4, further comprising using the patent's demographic information and the patient-specific 3D heart model to create a new 3D heart model for inclusion in the database of representative 3D heart models.

10. The method of claim 4, further comprising using the patent's demographic information and the patient-specific 3D heart model to adjusting a 3D heart model in the database of representative 3D heart models.

11. A computing system, comprising:
a memory having stored thereon a database of representative three-dimensional (3D) heart models; and
a processor coupled to the memory and configured with processor-executable instructions to perform operations comprising:
selecting a 3D heart electrical conduction model from a database of representative 3D heart models based on patient demographic information, the selected 3D heart electrical conduction model including a 3D surface model,
generating a patient-specific 3D localization of an arrhythmia based on the selected 3D electrical conduction model and electrocardiographic (ECG) data;
generating a patient-specific cardiac activation map based the 3D electrical conduction model and ECG data;
merging the patient-specific 3D localization of the arrhythmia and the 3D surface model to generate a 3D arrhythmia activation surface model; and
displaying the patient-specific 3D localization of the arrhythmia and the patient-specific cardiac activation map for use in a medical procedure.

12. The computing system of claim 11, wherein the patient demographic information includes one or more of the patient's gender, age, weight, height, body mass index, waist circumference, chest circumference, or underlying etiology.

13. The computing system of claim 11, wherein the processor is configured with processor-executable instructions to perform operations further comprising generating a patient-specific 3D heart model from the selected 3D heart model by:
obtaining a 3D image of ECG electrodes on the patient's torso; and
merging the 3D image of the patient's torso with the selected 3D heart model.

14. The computing system of claim 13, wherein the processor is configured with processor-executable instructions to perform operations such that merging the patient-specific 3D localization of the arrhythmia and the 3D surface model to generate a 3D arrhythmia activation surface model comprises aligning locations of ECG electrodes used in generating patient-specific electrical conduction map of a patient's heart with the ECG electrodes within the 3D image.

15. The computing system of claim 14, wherein the processor is configured with processor-executable instructions to perform operations further comprising combining ECG data obtained with 12 ECG electrodes with the patient specific 3D heart model using an inverse solution calculation to generate a localization point of the arrhythmia activation in a heartbeat.

16. The computing system of claim 14, wherein the arrhythmia is a ventricular arrhythmia.

17. The computing system of claim 16, wherein the ventricular arrhythmia is a pre-ventricular contraction (PVC).

18. The computing system of claim 16, wherein the ventricular arrhythmia is a ventricular tachycardia.

19. The computing system of claim 14, wherein the processor is configured with processor-executable instructions to perform operations further comprising using the patent's demographic information and the patient-specific 3D heart model to create a new 3D heart model for inclusion in the database of representative 3D heart models.

20. The computing system of claim 14, wherein the processor is configured with processor-executable instructions to perform operations further comprising using the patent's demographic information and the patient-specific 3D heart model to adjusting a 3D heart model in the database of representative 3D heart models.
